(19) United States
(12) Patent Application Publication
(10) Pub. No.: US 2007/0203178 A1
(43) Pub. Date: Aug. 30, 2007

(54) CRYSTALLINE SOLVATES OF APIXABAN

(76) Inventors: Mary F. Malley, Lawrenceville, NJ (US); Carolyn Jane Pommier, Somerset, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/678,206

(22) Filed: Feb. 23, 2007

Related U.S. Application Data
(63) Continuation-in-part of application No. 11/235,510, filed on Sep. 26, 2005.
(60) Provisional application No. 60/613,938, filed on Sep. 28, 2004.
(60) Provisional application No. 60/688,999, filed on Jun. 9, 2005.

(51) Int. Cl. A61K 31/4745; C07D 471/02

(52) U.S. Cl. 514/303; 546/118

(57) ABSTRACT

The present invention generally relates to crystalline dimethyl formamide and formamide solvates of apixaban, designated as Forms DMF-5 and FA-2, which are useful for preparing crystalline apixaban hydrate and neat forms.

### CROSS-REFERENCE TO RELATED APPLICATIONS

This application is a continuation-in-part of U.S. Ser. No. 11/235,510, filed Sep. 26, 2005, which claims priority from U.S. Provisional Application Ser. No. 60/613,938, filed Sep. 28, 2004, and U.S. Provisional Application Ser. No. 60/688,999, filed Jun. 9, 2005, each of which is fully incorporated by reference herein.

### FIELD OF THE INVENTION

The present invention provides novel crystalline dimethyl formamide and formamide solvates of apixaban and processes of their preparation.

### BACKGROUND OF THE INVENTION

U.S. Patent Publication No. 2003/0191115, which is herein incorporated by reference in its entirety, discloses a series of coagulation Factor Xa inhibitors including 1-(4-methoxyphenyl)-7-oxo-6-[4-(2-oxo-1-piperidinyl)phenyl]-4,5,6,7-tetrahydro-1H-pyrazolo[3,4-c]pyridine-3-carboxamide (also known as apixaban). Apixaban is a highly potent and selective inhibitor of Factor Xa and thus is useful in preventing or treating thromboembolic disorders.

There exists a need for crystalline forms which may exhibit desirable and beneficial chemical and physical properties. There also exists a need for reliable and reproducible methods for the manufacture, purification, and formulation of apixaban to permit its feasible commercialization. We have discovered novel crystalline dimethyl formamide and formamide solvates of apixaban, designated as Forms DMF-5 and FA-2, which are useful for preparing crystalline apixaban hydrate and neat forms.

### SUMMARY OF THE INVENTION

The present invention encompasses crystalline solvates of 1-(4-methoxyphenyl)-7-oxo-6-[4-(2-oxo-1-piperidinyl)phenyl]-4,5,6,7-tetrahydro-1H-pyrazolo[3,4-c]pyridine-3-carboxamide (hereinafter referred to as apixaban). The formula of apixaban is shown below.

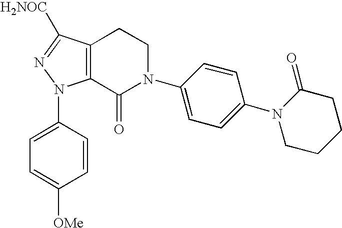

In particular, the present invention provides crystalline dimethyl formamide solvate and formamide solvate of apixaban. Embodiments of these crystalline solvates include those described and characterized herein as Forms DMF-5 and FA-2.

These and other features of the invention will be set forth in the expanded form as the disclosure continues.

### BRIEF DESCRIPTION OF THE DRAWINGS

The invention is illustrated by reference to the accompanying drawings described below.

FIG. 1 shows simulated (−50° C.) and observed (room temperature) powder X-ray diffraction patterns (CuKα λ=1.5418 Å) of Form DMF-5 of crystalline apixaban.

FIG. 2 shows simulated (−70° C.) and observed (room temperature) powder X-ray diffraction patterns (CuKα λ=1.5418 Å) of Form FA-2 of crystalline apixaban.

FIG. 3 shows differential scanning calorimetry thermogram of Form DMF-5 of crystalline apixaban.

FIG. 4 shows differential scanning calorimetry thermogram of Form FA-2 of crystalline apixaban.

FIG. 5 shows thermogravimetric analysis curve of Form DMF-5 of crystalline apixaban.

FIG. 6 shows thermogravimetric analysis curve of Form FA-2 of crystalline apixaban.

### DETAILED DESCRIPTION OF THE INVENTION

All examples provided in the definitions as well as in other portions of this application are not intended to be limiting, unless stated.

Definitions

The names used herein to characterize a specific form, e.g. “DMF-5”, should not be considered limiting with respect to any other substance possessing similar or identical physical and chemical characteristics, but rather it should be understood that these designations are mere identifiers that should be interpreted according to the characterization information also presented herein.

As used herein, “solvate” means a physical association of a compound with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvate may comprise either a stoichiometric or nonstoichiometric amount of the solvent molecules. For example, a solvate with a nonstoichiometric amount of solvent molecules may result from partial loss of solvent from the solvate. “Solvate” encompasses both solution-phase and isolable solvates. Exemplary solvates include hydrates, ethanolates, methanolates, isopropanolates and the like. Methods of solvation are generally known in the art.

As used herein, “polymorph” refers to crystalline forms having the same chemical composition but different spatial arrangements of the molecules, atoms, and/or ions forming the crystal.

As used herein “amorphous” refers to a solid form of a molecule, atom, and/or ions that is not crystalline. An amorphous solid does not display an X-ray diffraction pattern with sharp maxima.

The present invention provides, at least in part, a crystalline form of apixaban as a novel material. In certain preferred embodiments, crystalline forms of apixaban are in substantially pure form.

As used herein, “substantially pure,” when used in reference to a crystalline form, means a compound having a purity greater than 90 weight %, including greater than 90, 91, 92, 93, 94, 95, 96, 97, 98 and 99 weight %, and also including equal to about 100 weight % of the compound, based on the weight of the compound. The remaining material comprises other form(s) of the compound, and/or reaction impurities and/or processing impurities arising from its preparation. For example, a crystalline form of the compound may be deemed substantially pure in that it has a purity greater than 90 weight %, as measured by means that are at this time known and generally accepted in the art, where the remaining less than 10 weight % of material comprises other form(s) of the compound and/or reaction impurities and/or processing impurities.

Samples of the crystalline forms may be provided with substantially pure phase homogeneity, indicating the presence of a dominant amount of a single crystalline form and optionally minor amounts of one or more other crystalline forms. The presence of more than one crystalline form in a sample may be determined by techniques such as powder X-ray diffraction (PXRD) or solid state nuclear magnetic resonance spectroscopy (SSNMR). For example, the presence of extra peaks in the comparison of an experimentally measured PXRD pattern with a simulated PXRD pattern may indicate more than one crystalline form in the sample. The simulated PXRD may be calculated from single crystal X-ray data. see Smith, D. K., “A FORTRAN Program for Calculating X-Ray Powder Diffraction Patterns,” Lawrence Radiation Laboratory, Livermore, Calif., UCRL-7196, April 1963.

Procedures for the preparation of crystalline forms are known in the art. The crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Ind., 1999.

For crystallization techniques that employ solvent, the choice of solvent or solvents is typically dependent upon one or more factors, such as solubility of the compound, crystallization technique, and vapor pressure of the solvent. Combinations of solvents may be employed; for example, the compound may be solubilized into a first solvent to afford a solution, followed by the addition of an antisolvent to decrease the solubility of the compound in the solution and to afford the formation of crystals. An “antisolvent” is a solvent in which the compound has low solubility. Suitable solvents for preparing crystals include polar and nonpolar solvents.

In one method to prepare crystals, a compound is suspended and/or stirred in a suitable solvent to afford a slurry, which may be heated to promote dissolution. The term “slurry,” as used herein, means a saturated solution of the compound, which may also contain an additional amount of the compound to afford a heterogeneous mixture of the compound and a solvent at a given temperature. Suitable solvents in this regard include, for example, polar aprotic solvents and polar protic solvents, and nonpolar solvents, and mixtures of two or more of these, as disclosed herein.

Suitable polar aprotic solvents include, for example, dichloromethane (CH₂Cl₂ or DCM), tetrahydrofuran (THF), acetone, methyl ethyl ketone (MEK), dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide (FA), N-methylacetamide, N-methylformamide, acetonitrile (ACN or MeCN), dimethylsulfoxide (DMSO), propionitrile, ethyl formate, methyl acetate (MeOAc), ethyl acetate (EtOAc), isopropyl acetate (IpOAc), butyl acetate (BuOAc), t-butyl acetate, hexachloroacetone, dioxane, sulfolane, N,N-dimethylpropionamide, nitromethane, nitrobenzene and hexamethylphosphoramide.

Suitable polar protic solvents include, for example, alcohols and glycols, such as H₂O, methanol, ethanol, 1-propanol, 2-propanol, isopropanol (IPA), 1-butanol (1-BuOH), 2-butanol (2-BuOH), i-butyl alcohol, t-butyl alcohol, 2-nitroethanol, 2-fluoroethanol, 2,2,2-trifluoroethanol, ethylene glycol, 2-methoxyethanol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol and methyl t-butyl ether (MTBE).

Preferred solvents include, for example, acetone, H₂O, MeCN, CH₂Cl₂, THF, ethanol, n-BuOH, 2-BuOH, IPA, EtOAc, DMF and formamide.

Other solvents suitable for the preparation of slurries of the compound, in addition to those exemplified above, would be apparent to one skilled in the art, based on the present disclosure.

Seed crystals may be added to any crystallization mixture to promote crystallization. As will be clear to the skilled artisan, seeding is used as a means of controlling growth of a particular crystalline form or as a means of controlling the particle size distribution of the crystalline product. Accordingly, calculation of the amount of seeds needed depends on the size of the seed available and the desired size of an average product particle as described, for example, in “Programmed cooling of batch crystallizers,” J. W. Mullin and J. Nyvlt, Chemical Engineering Science, 1971, 26, 369-377. In general, seeds of small size are needed to effectively control the growth of crystals in the batch. Seeds of small size may be generated by sieving, milling, or micronizing of larger crystals, or by micro-crystallization of solutions. Care should be taken that milling or micronizing of crystals does not result in any change in crystallinity of the desired crystal form or form conversions (i.e. change to amorphous or to another polymorph).

A cooled mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as SSNMR, DSC, PXRD, or the like, to assure formation of the preferred crystalline form of the product. The resulting crystalline form is typically produced in an amount of greater than about 70 weight % isolated yield, but preferably greater than 90 weight % based on the weight of the compound originally employed in the crystallization procedure. The product may be co-milled or passed through a mesh screen to de-lump the product, if necessary.

Crystalline forms may be prepared directly from the reaction medium of the final process step for preparing a compound. This may be achieved, for example, by employing in the final process step a solvent or mixture of solvents from which the compound may be crystallized. Alternatively, crystalline forms may be obtained by distillation or solvent addition techniques. Suitable solvents for this purpose include any of those solvents described herein, including protic polar solvents, such as alcohols, and aprotic polar solvents, such as ketones.

By way of general guidance, the reaction mixture may be filtered to remove any undesired impurities, inorganic salts, and the like, followed by washing with reaction or crystallization solvent. The resulting solution may be concentrated to remove excess solvent or gaseous constituents. If distillation is employed, the ultimate amount of distillate collected may vary, depending on process factors including, for example, vessel size, stirring capability, and the like. By way of general guidance, the reaction solution may be distilled to about 1/10 the original volume before solvent replacement is carried out. The reaction may be sampled and assayed to determine the extent of the reaction and the wt % product in accordance with standard process techniques. If desired, additional reaction solvent may be added or removed to optimize reaction concentration. Preferably, the final concentration is adjusted to about 50 wt % at which point a slurry typically results.

It may be preferable to add solvents directly to the reaction vessel without distilling the reaction mixture. Preferred solvents for this purpose are those which may ultimately participate in the crystalline lattice, as discussed above in connection with solvent exchange. Although the final concentration may vary depending on desired purity, recovery and the like, the final concentration of free base I in solution is preferably about 4% to about 7%. The reaction mixture may be stirred following solvent addition and simultaneously warmed. By way of illustration, the reaction mixture may be stirred for about 1 hour while warming to about 70° C. The reaction is preferably filtered hot and washed with either the reaction solvent, the solvent added or a combination thereof. Seed crystals may be added to any crystallization solution to initiate crystallization.

The forms may be characterized and distinguished using single crystal X-ray diffraction, which is based on unit cell and intensity measurements of a single crystal of a form at a fixed analytical temperature. A detailed description of unit cells and intensity analysis is provided in Stout & Jensen, X-Ray Structure Determination: A Practical Guide, Macmillan Co., New York (1968), Chapter 3, which is herein incorporated by reference. Alternatively, the unique arrangement of atoms in spatial relation within the crystalline lattice may be characterized according to the observed fractional atomic coordinates. See Stout & Jensen reference for experimental determination of fractional coordinates for structural analysis. Another means of characterizing the crystalline structure is by powder X-ray diffraction analysis in which the experimental or observed diffraction profile is compared to a simulated profile representing pure powder material, both at the same analytical temperature, and measurements for the subject form characterized as a series of 2θ values and intensities.

Other means of characterizing the form may be used, such as solid state nuclear magnetic resonance (SSNMR), differential scanning calorimetry (DSC) and thermogravimetric analysis (TGA). These parameters may also be used in combination to characterize the subject form.

One of ordinary skill in the art will appreciate that an X-ray diffraction pattern may be obtained with a measurement error that is dependent upon the measurement conditions employed. In particular, it is generally known that intensities in a X-ray diffraction pattern may fluctuate depending upon measurement conditions employed and the shape or morphology of the crystal. It should be further understood that relative intensities may also vary depending upon experimental conditions and, accordingly, the exact order of intensity should not be taken into account. Additionally, a measurement error of diffraction angle for a conventional X-ray diffraction pattern is typically about 0.2° or less, preferably about 0.1° (as discussed hereinafter), and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art.

The term “negligible weight loss,” as employed herein, as characterized by TGA indicates the presence of a neat (non-solvated) crystal form. The term “negligible % water uptake,” as employed herein, as characterized by moisture-sorption isotherm indicates that the form tested is non-hygroscopic.

Apixaban may be prepared using methods known to the skilled artisan of organic synthesis, as well as methods taught in commonly assigned U.S. Application Publication Nos. 2003/0191115, US20030181466, and US2006/0069085 and US20060069258, the disclosures of which are hereby incorporated herein by reference, in their entireties.

In one aspect of the present invention, crystalline dimethyl formamide solvate Form DMF-5 (Chemical formula: C₂₅H₂₅N₅O₄.C₃H₇NO) of apixaban may be characterized by unit cell parameters substantially equal to the following:

| | |
|---|---|
| Cell dimensions: | a = 6.307(1) Å |
| | b = 31.385(6) Å |
| | c = 13.635(3) Å |
| | α = 90° |
| | β = 90.15(3)° |
| | γ = 90° |
| | 9 |
| Space group P2₁/n | |
| Molecules/asymmetric unit 1 | |

wherein the crystalline form is at about +25° C.

In a different aspect, Form DMF-5 may be characterized by unit cell parameters substantially equal to the following:

| | |
|---|---|
| Cell dimensions: | a = 6.288(1) Å |
| | b = 31.242(6) Å |
| | c = 13.493(3) Å |
| | α = 90° |
| | β = 90.48(3)° |
| | γ = 90° |
| Space group P2₁/n | |
| Molecules/asymmetric unit 1 | |

wherein the crystalline form is at about −50° C.

In a different aspect, Form DMF-5 may be characterized by fractional atomic coordinates substantially as listed in Table 2.

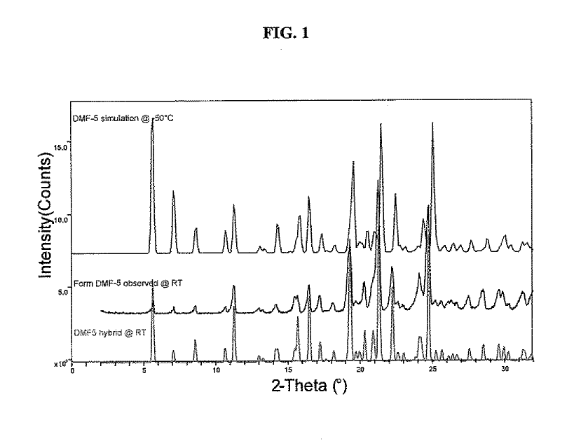

In a different aspect, Form DMF-5 may be characterized by a powder X-ray diffraction pattern (FIG. 1) comprising the following 2θ values (CuKα λ=1.5418 Å): 5.6±0.1, 7.1±0.1, 8.6±0.1, 14.3±0.1, 20.3±0.1, and 24.7±0.1, at about 25° C.

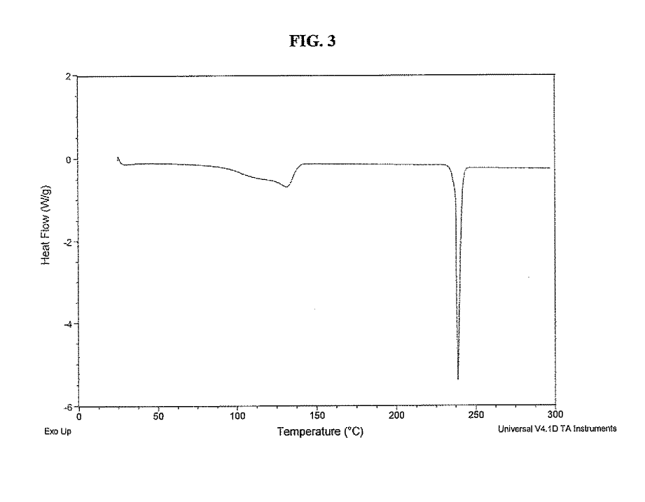

In a different aspect, Form DMF-5 may be characterized by a differential scanning calorimetry thermogram (FIG. 3) having a broad endotherm from ca. 70° C. to ca. 150° C., at higher temperatures other events may ensue.

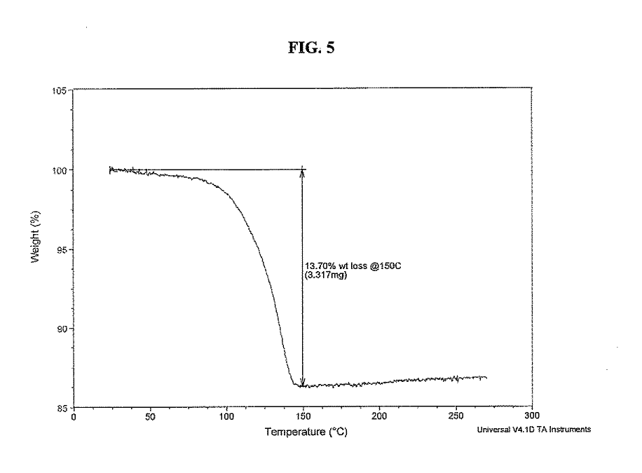

In a different aspect, Form DMF-5 may be characterized by a thermal gravimetric analysis curve (FIG. 5) having a weight loss of ca. 13.7% up to ca. 150° C.

In one aspect of the present invention, crystalline formamide solvate Form FA-2 (Chemical formula: C₂₅H₂₅N₅O₄.CH₃NO) of apixaban may be characterized by unit cell parameters substantially equal to the following:

| | |
|---|---|
| Cell dimensions: | a = 6.304(1) Å |
| | b = 30.164(3) Å |
| | c = 12.960(3) Å |
| | α = 90° |
| | β = 92.17(3)° |
| | γ = 90° |
| Space group P2₁/n | |
| Molecules/asymmetric unit 1 | |

wherein the crystalline form is at about −70° C.

In a different aspect, Form FA-2 may be characterized by fractional atomic coordinates substantially as listed in Table 3.

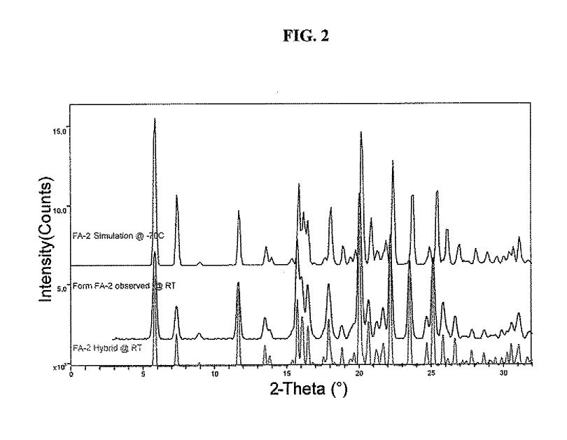

In a different aspect, Form FA-2 may be characterized by a powder X-ray diffraction pattern (FIG. 2) comprising the following 2θ values (CuKα λ=1.5418 Å): 8.9±0.1, 11.7±0.1, 15.8±0.1, 16.5±0.1, and 25.1±0.1, at about 25° C.

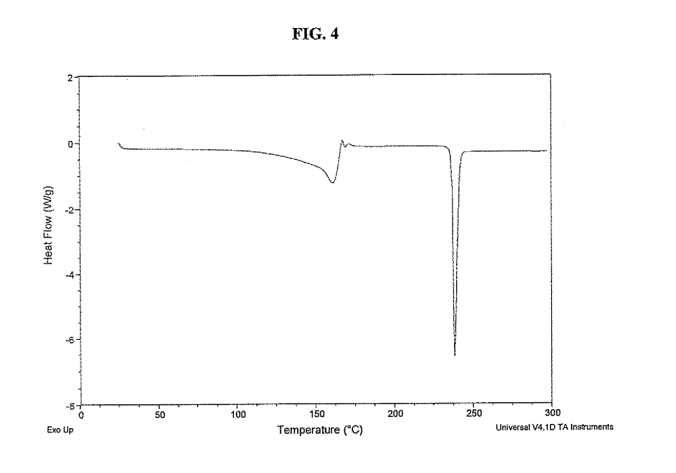

In a different aspect, Form FA-2 may be characterized by a differential scanning calorimetry thermogram (FIG. 4) having a broad endotherm from ca. 75° C. to ca. 170° C., at higher temperatures other events may ensue.

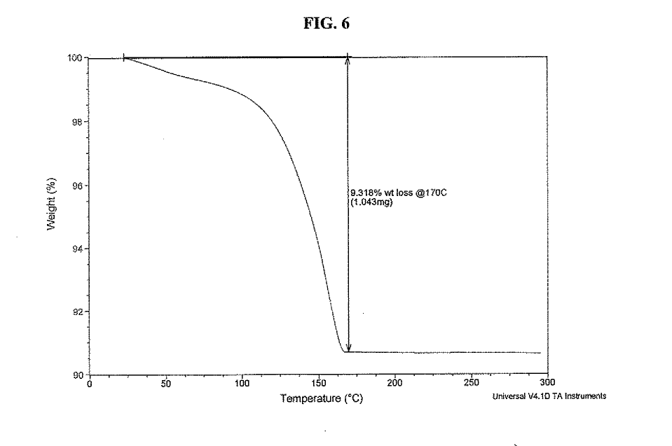

In a different aspect, Form FA-2 may be characterized by a thermal gravimetric analysis curve (FIG. 6) having a weight loss ca. 8.9% up to ca. 170° C.

Other features of the invention will become apparent in the course of the following descriptions of exemplary embodiments which are given for illustration of the invention and are not intended to be limiting thereof.

### EXAMPLES

### Example 1

Preparation of Form DMF-5

900 mg of dried crude apixaban was agitated for 16-24 hours as a slurry in anhydrous DMF. There was sufficient amount of apixaban such that solid was present at all times. The slurry was dried by passing a stream of air over the sample in the vial to obtain Form DMF-5.

### Example 2

Preparation of Form FA-2

1 g of dried crude apixaban was slurried in formamide. There was sufficient amount of apixaban such that solid was present at all times. After four hours, the sample was observed to have changed morphology. The slurry was filtered and washed with isopropyl alcohol, then dried on the filter by allowing air to pass through the sample at room temperature for at least 30 minutes to obtain Form FA-2.

X-ray powder diffraction (PXRD) data were obtained using a Bruker C2 GADDS (General Area Detector Diffraction System). The radiation was Cu Kα (40 KV, 50 mA). The sample-detector distance was 15 cm. Powder samples were placed in sealed glass capillaries of 1 mm or less in diameter; the capillary was rotated during data collection. Data were collected for 3≦2θ≦35°with a sample exposure time of at least 2000 seconds. The resulting two-dimensional diffraction arcs were integrated to create a traditional 1-dimensional PXRD pattern with a step size of 0.02 degrees 2θ in the range of 3 to 35 degrees 2θ.

Characteristic diffraction peak positions (degrees 2θ±0.1) at room tempeature or at a specified temperature were determined based on a high quality pattern collected with a diffractometer (CuKα) with a spinning capillary with 2θ calibrated with a NIST or other suitable standard.

Single crystal X-ray data were collected on a Bruker-Nonius CAD4 serial diffractometer (Bruker Axs, Inc., Madison Wis.). Unit cell parameters were obtained through least-squares analysis of the experimental diffractometer settings of 25 high-angle reflections. Intensities were measured using Cu Kα radiation (λ=1.5418 Å) at a constant temperature with the θ-2θ variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (λ=1.5418 Å). Indexing and processing of the measured intensity data were carried out with the HKL2000 software package in the Collect program suite R. Hooft, Nonius B. V. (1998). When indicated, crystals were cooled in the cold stream of an Oxford cryogenic system during data collection.

The structures were solved by direct methods and refined on the basis of observed reflections using either the SDP software package SDP, Structure Determination Package, Enraf-Nonius, Bohemia, N.Y.) with minor local modifications or the crystallographic package, MAXUS (maXus solution and refinement software suit: S. Mackay, C. J. Gilmore, C. Edwards, M. Tremayne, N. Stewart, and K. Shankland. maXus is a computer program for the solution and refinement of crystal structures from diffraction data.

The derived atomic parameters (coordinates and temperature factors) were refined through full matrix least-squares. The function minimized in the refinements was Σ_{w}(|Fₒ|−|F_{c}|)². R is defined as Σ||F|−|F||/Σ|Fₒ|while R_{w}=[Σ_{w}(|Fₒ|−|F_{c}|)²/Σ_{w} |Fₒ|²]^{1/2} where w is an appropriate weighting function based on errors in the observed intensities. Difference maps were examined at all stages of refinement. Hydrogen atoms were introduced in idealized positions with isotropic temperature factors, but no hydrogen parameters were varied.

“Hybrid” simulated powder X-ray patterns were generated as described in the literature (Yin. S.; Scaringe, R. P.; DiMarco, J.; Galella, M. and Gougoutas, J. Z., American Pharmaceutical Review, 2003, 6(2), 80). The room temperature cell parameters were obtained by performing a cell refinement using the CellRefine.xls program. Input to the program includes the 2-theta position of ca. 10 reflections, obtained from the experimental room temperature powder pattern; the corresponding Miller indices, hkl, were assigned based on the single-crystal data collected at low temperature. A new (hybrid) PXRD was calculated (by either of the software programs, Alex or LatticeView) by inserting the molecular structure determined at low temperature into the room temperature cell obtained in the first step of the procedure. The molecules are inserted in a manner that retains the size and shape of the molecule and the position of the molecules with respect to the cell origin, but, allows intermolecular distances to expand with the cell.

Differential scanning calorimetry (DSC) experiments were performed in a TA Instruments™ model Q1000. The sample (about 2-6 mg) was weighed in an aluminum pan and recorded accurately recorded to a hundredth of a milligram, and transferred to the DSC. The instrument was purged with nitrogen gas at 50 mL/min. Data were collected between room temperature and 300° C. at 10° C./min heating rate. The plot was made with the endothermic peaks pointing down.

Thermal gravimetric analysis (TGA) experiments were performed in a TA Instruments™ model Q500. The sample (about 10-30 mg) was placed in a platinum pan previously tared. The weight of the sample was measured accurately and recorded to a thousand of a milligram by the instrument The furnace was purged with nitrogen gas at 100 mL/min. Data were collected between room temperature and 300° C. at 10° C./min heating rate.

The unit cell data and other properties for DMF-5 and FA-2 are tabulated in Tables 1a and 1b. The unit cell parameters were obtained from single crystal X-ray crystallographic analysis.

TABLE 1a
Unit Cell Parameters

| | T | | | | | | |
|---|---|---|---|---|---|---|---|
| Form | (° C.) | a(Å) | b(Å) | c(Å) | α° | β° | γ° |
| DMF-5 | +25 | 6.307(1) | 31.385(6) | 13.635(3) | 90 | 90.15(3) | 90 |
| DMF-5 | −50 | 6.288(1) | 31.242(6) | 13.493(3) | 90 | 90.48(3) | 90 |
| FA-2 | −70 | 6.304(1) | 31.164(6) | 12.960(3) | 90 | 92.17(3) | 90 |

TABLE 1b
Unit Cell Parameters (continued)

| Form | T(° C.) | Vₘ(Å³) | Z′ | SG | R | Solvent Sites for Z′ |
|---|---|---|---|---|---|---|
| DMF-5 | +25 | 675 | 1 | P2₁/n | — | 1 DMF |
| DMF-5 | −50 | 663 | 1 | P2₁/n | 0.05 | 1 DMF |
| FA-2 | −70 | 616 | 1 | P2₁/n | 0.06 | 1 formamide |

Notes for Tables:
T is the temperature for the crystallographic data.
Z′ is the number of molecules of Compound (I) in each asymmetric unit (not unit cell).
Vₘ is the molar volume, V(unit cell)/(Z drug molecules per cell).
SG is the crystallographic space group.
R is the R-factor (measure of the quality of the refinement).
At 25° C., only the cell for DMF-5 was determined. There was no structure refinement.

The fractional atomic coordinates for DMF-5 and FA-2 are tabulated in Tables 2 and 3. Numbers in parentheses are estimated standard deviations in the least significant digits.

TABLE 2
Positional Parameters and Their Estimated Standard Deviations for Form
DMF-5 at −50° C.

| Atom | x | y | z | B (iso) |
|---|---|---|---|---|
| O8 | 0.4159(4) | 0.32785(11) | 0.1211(3) | 3.0 |
| O13 | 1.0946(5) | 0.30953(14) | −0.1984(3) | 4.6 |
| O16 | −0.0331(6) | 0.13699(12) | 0.0909(4) | 5.0 |
| O25 | −0.3784(4) | 0.46251(11) | 0.3879(3) | 2.9 |
| O99 | 0.6824(9) | 0.43789(19) | −0.1070(5) | 9.1 |
| N1 | 0.4031(5) | 0.24482(15) | 0.0058(3) | 2.2 |
| N2 | 0.3577(5) | 0.20391(15) | −0.0165(3) | 2.4 |
| N7 | 0.0884(5) | 0.31013(14) | 0.1801(3) | 2.0 |
| N17 | 0.1796(6) | 0.12587(14) | −0.0398(3) | 3.1 |
| N24 | −0.0514(5) | 0.47718(14) | 0.3273(3) | 1.9 |
| N97 | 0.3533(7) | 0.44612(18) | −0.0402(4) | 4.6 |
| C3 | 0.1954(6) | 0.19264(16) | 0.0415(4) | 2.0 |
| C4 | 0.1320(6) | 0.22752(17) | 0.1002(4) | 2.0 |
| C5 | −0.0412(6) | 0.23487(17) | 0.1737(4) | 2.7 |
| C6 | −0.0994(6) | 0.28214(16) | 0.1723(4) | 2.4 |
| C8 | 0.2671(6) | 0.30241(18) | 0.1258(4) | 2.4 |
| C9 | 0.2680(6) | 0.26020(17) | 0.0769(4) | 1.9 |
| C10 | 0.5763(6) | 0.26354(19) | −0.0468(4) | 2.1 |
| C11 | 0.7439(6) | 0.23662(18) | −0.0738(4) | 2.9 |
| C12 | 0.9123(6) | 0.25335(19) | −0.1262(4) | 2.7 |
| C13 | 0.9180(6) | 0.2962(2) | −0.1498(4) | 2.8 |
| C14 | 0.7499(7) | 0.32302(18) | −0.1249(4) | 3.1 |
| C15 | 0.5774(6) | 0.30573(19) | −0.0739(4) | 2.6 |
| C16 | 0.1045(7) | 0.14947(17) | 0.0344(4) | 2.8 |
| C18 | 0.0537(6) | 0.35155(17) | 0.2201(4) | 1.8 |
| C19 | −0.1353(6) | 0.37334(19) | 0.1993(4) | 2.5 |
| C20 | −0.1706(6) | 0.41382(17) | 0.2357(4) | 2.2 |
| C21 | −0.0211(6) | 0.43419(17) | 0.2940(4) | 2.0 |
| C22 | 0.1673(6) | 0.41282(17) | 0.3164(4) | 2.2 |
| C23 | 0.2035(6) | 0.37193(18) | 0.2797(4) | 2.5 |
| C25 | −0.2365(6) | 0.48867(17) | 0.3726(4) | 2.0 |
| C26 | −0.2698(6) | 0.53461(17) | 0.4002(4) | 2.9 |
| C27 | −0.0671(7) | 0.56071(18) | 0.4142(4) | 3.5 |
| C28 | 0.0782(7) | 0.55234(17) | 0.3289(4) | 3.2 |
| C29 | 0.1348(6) | 0.50631(17) | 0.3225(4) | 3.1 |
| C30 | 1.1022(9) | 0.3529(2) | −0.2299(5) | 5.3 |
| C95 | 0.2948(14) | 0.4813(3) | −0.0995(7) | 9.4 |
| C96 | 0.2090(13) | 0.4318(3) | 0.0342(7) | 9.6 |
| C98 | 0.5418(12) | 0.4271(3) | −0.0482(7) | 8.6 |
| H17F | 0.3062 | 0.1376 | −0.0873 | |
| H16F | 0.1203 | 0.0937 | −0.0514 | |

Occupancies are 1. unless otherwise indicated.

TABLE 3
Positional Parameters and Their Estimated Standard
Deviations for Form FA-2 at-70° C.

| | | | | | Occu- |
|---|---|---|---|---|---|
| Atom | x | y | z | B (iso) | pancy* |
| O8 | 0.7085(4) | 0.33488(8) | −0.09691(18) | 3.4 | |
| O16 | 0.2451(5) | 0.13916(8) | −0.1401(2) | 4.7 | |
| O30 | 1.3379(4) | 0.32137(10) | −0.4451(2) | 5.0 | |
| O25 | −0.0372(4) | 0.46958(8) | 0.2097(2) | 4.0 | |
| O98 | 0.1373(13) | 0.04699(18) | −0.0709(4) | 8.6 | 0.7 |
| N1 | 0.6862(4) | 0.25052(9) | −0.2200(2) | 2.6 | |
| N2 | 0.6434(4) | 0.20769(9) | −0.2409(2) | 2.9 | |
| N7 | 0.3787(4) | 0.31797(8) | −0.0399(2) | 2.5 | |
| N24 | 0.2890(4) | 0.48485(9) | 0.1527(2) | 3.0 | |
| N28 | 0.5066(5) | 0.12354(10) | −0.2495(3) | 4.4 | |
| C3 | 0.4795(5) | 0.19665(11) | −0.1833(2) | 2.7 | |
| C4 | 0.4109(5) | 0.23323(11) | −0.1263(2) | 2.5 | |
| C5 | 0.2363(5) | 0.24116(11) | −0.0541(3) | 2.7 | |
| C6 | 0.1873(5) | 0.28996(11) | −0.0529(3) | 3.0 | |
| C8 | 0.5559(5) | 0.30989(11) | −0.0957(2) | 2.4 | |
| C9 | 0.5472(5) | 0.26678(11) | −0.1494(2) | 2.4 | |
| C10 | 0.8523(5) | 0.27082(11) | −0.2758(2) | 2.5 | |
| C11 | 0.8382(5) | 0.31422(12) | −0.3066(3) | 3.1 | |
| C12 | 1.0005(6) | 0.33274(12) | −0.3635(3) | 3.3 | |
| C13 | 1.1707(6) | 0.30678(14) | −0.3897(3) | 3.5 | |
| C14 | 1.1796(6) | 0.26295(13) | −0.3598(3) | 3.5 | |
| C15 | 1.0221(5) | 0.24467(12) | −0.3028(3) | 2.9 | |
| C16 | 0.3992(6) | 0.15078(12) | −0.1887(3) | 3.2 | |
| C18 | 0.3572(5) | 0.35994(11) | 0.0100(2) | 2.4 | |
| C19 | 0.5117(5) | 0.37596(11) | 0.0777(3) | 2.9 | |
| C20 | 0.4884(5) | 0.41659(11) | 0.1253(3) | 3.1 | |
| C21 | 0.3080(5) | 0.44216(11) | 0.1047(3) | 2.7 | |
| C22 | 0.1532(5) | 0.42625(12) | 0.0362(3) | 3.0 | |
| C23 | 0.1760(5) | 0.38540(12) | −0.0102(3) | 2.8 | |
| C25 | 0.1144(6) | 0.49547(12) | 0.2047(3) | 3.2 | |
| C26 | 0.1004(7) | 0.54032(13) | 0.2541(3) | 4.5 | |
| C27 | 0.3053(8) | 0.56397(16) | 0.2716(5) | 6.7 | |
| C28 | 0.4457(8) | 0.55891(16) | 0.1858(5) | 6.7 | |
| C29 | 0.4785(7) | 0.51338(13) | 0.1540(3) | 4.6 | |
| C30 | 1.3367(8) | 0.36582(16) | −0.4783(4) | 5.9 | |
| N97 | 0.2605(8) | −0.01789(16) | −0.0203(4) | 5.4 | 0.7 |
| C98 | 0.2708(14) | 0.0200(3) | −0.0567(5) | 6.4 | 0.7 |
| H98 | 0.4054 | 0.0285 | −0.0760 | | 0.7 |
| H28F | 0.6387 | 0.1351 | −0.2922 | | |
| H29F | 0.4538 | 0.0892 | −0.2590 | | |
| H31F | 0.3920 | −0.0392 | −0.0052 | | 0.7 |
| H30F | 0.0991 | −0.0295 | −0.0047 | | 0.7 |

*Occupancies are 1. unless otherwise indicated.

Numerous modifications and variations of the present invention are possible in light of the above teachings. It is therefore to be understood that within the scope of the appended claims, the invention may be practiced otherwise than as specifically described herein.

What is claimed is:

1. Crystalline dimethyl formamide solvate Form DMF-5 of apixaban, which is characterized by unit cell parameters substantially equal to the following:

| | |
|---|---|
| Cell dimensions: | a = 6.307(1) Å |
| | b = 31.385(6) Å |
| | c = 13.635(3) Å |
| | α = 90° |
| | β = 90.15(3)° |
| | γ = 90° |
| Space group P2₁/n | |
| Molecules/asymmetric unit 1 | |

wherein said crystalline form is at about +25° C.

2. Form DMF-5 according to claim 1, which is characterized by fractional atomic coordinates substantially as listed in Table 2.

3. Form DMF-5 according to claim 1, which is characterized by a powder X-ray diffraction pattern substantially in accordance with that shown in FIG. 1.

4. Form DMF-5 according to claim 1 having a powder X-ray diffraction pattern comprising the following 2θ values (CuKα λ=1.5418 Å) 5.6±0.1, 7.1±0.1, 8.6±0.1, 14.3±0.1, 20.3±0.1, and 24.7±0.1, at about 25° C.

5. Crystalline formamide solvate Form FA-2 of apixaban, which is characterized by unit cell parameters substantially equal to the following:

| | |
|---|---|
| Cell dimensions: | a = 6.304(1) Å |
| | b = 30.164(3) Å |
| | c = 12.960(3) Å |
| | α = 90° |
| | β = 92.17(3)° |
| | γ = 90° |
| Space group P2₁/n | |
| Molecules/asymmetric unit 1 | |

wherein said crystalline form is at about −70° C.

6. Form FA-2 according to claim 5, which is characterized by fractional atomic coordinates substantially as listed in Table 3.

7. Form FA-2 according to claim 5, which is characterized by a powder X-ray diffraction pattern substantially in accordance with that shown in FIG. 2.

8. Form FA-2 according to claim 5 having a powder X-ray diffraction pattern comprising the following 2θ values (CuKα λ=1.5418 Å) 8.9±0.1, 11.7±0.1, 15.8±0.1, 16.5±0.1, and 25.1±0.1, at about 25° C.
